(19) United States
(12) Patent Application Publication
(10) Pub. No.: US 2020/0154730 A1
(43) Pub. Date: May 21, 2020

(54) High-Efficiency Processes for Protein and Oil Recovery from Thin Stillage

(71) Applicant: MARQUIS PROCAP SYSTEM LLC, Hennepin, IL (US)

(72) Inventors: Amanda Marquis, Hennepin, IL (US); Jason Marquis, Hennepin, IL (US)

(21) Appl. No.: 16/687,608

(22) Filed: Nov. 18, 2019

Related U.S. Application Data
(60) Provisional application No. 62/768,712, filed on Nov. 16, 2018.

(51) Int. Cl. A23J 1/00; B01D 21/01; B01D 21/00; B01D 17/04; B01D 21/26; C02F 9/00; A23J 1/12; A23J 1/18; A23K 10/38; A23K 20/147; C07K 14/415; C07K 14/39; C07K 1/14

(52) U.S. Cl. CPC A23J 1/005; C02F 1/004; B01D 21/0012; B01D 21/0027; B01D 17/047; B01D 21/262; C02F 9/00; A23J 1/125; A23J 1/18; A23K 10/38; A23K 20/147; C07K 14/415; C07K 14/39; C07K 1/145; B01D 21/01

(57) ABSTRACT

The present technology provides a process comprising: subjecting flocculated thin stillage comprising stillage solids and a polymer flocculent to a barrel screen to separate a liquid stream from a solids stream, wherein the solids stream comprises flocs of the polymer flocculant and the stillage solids; combining the solids stream with an effective amount of a demulsifier and/or inorganic particles to form a treated solids mixture; aging the treated solids mixture for a period of time at a temperature of 160 to 212° F. to form an aged solids mixture; and separating oil and water from the aged solids mixture to provide a high protein cake comprising at least 35% dry weight crude protein.

### CROSS-REFERENCE TO RELATED PATENT APPLICATIONS

This application claims the benefit of priority to U.S. Provisional Application No. 62/768,712, filed on Nov. 16, 2018, and which is incorporated herein by reference in its entirety for all purposes.

### FIELD

The present technology relates generally to processes for the recovery of grain proteins, yeast proteins, or grain and yeast proteins from thin stillage produced during the manufacture of ethanol from grain as well as processes for separating and recovering oil from the thin stillage. Compositions high in recovered grain and/or yeast protein as also provided.

### BACKGROUND

Traditional ethanol manufacturing processes utilize dry or wet milling of corn and/or other grains to provide a powder or powder slurry that is combined with water and yeast and is fermented to produce ethanol. The ethanol is distilled from the fermented mash and the non-fermentable solids and other residue left behind is referred to as stillage. The stillage is typically centrifuged to provide wet cake, which is a mixture of protein, oil, water, and unfermentable solids. The wet cake is dried to a low moisture product known as distillers dried grains with solubles (“DDGS”). The centrifugation of stillage also provides a liquid stream with both suspended and dissolved solids in water that is often split into two streams: one directed back to the ethanol production process and a second that is concentrated by evaporation (“a syrup stream”) and added to the wet cake to produce the DDGS. Although the DDGS may be sold for animal feed, overall, the treatment of ethanol stillage, especially thin stillage, is energy intensive and low value.

### SUMMARY

The present technology provides highly efficient processes to recover a product from thin stillage that is higher in proteins than traditional DDGS. The proteins may include grain proteins, yeast proteins, or both grain and yeast proteins. The high-protein product may also include less oil than traditional DDGS. The processes include subjecting flocculated thin stillage comprising stillage solids and a polymer flocculent to a barrel screen or air flotation process (e.g., DAF) to separate a liquid stream from a solids stream, wherein the solids stream comprises flocs of the polymer flocculant and the stillage solids; combining the solids stream with an effective amount of a demulsifier and/or inorganic particles to form a treated solids mixture; aging the treated solids mixture for a period of time at a temperature of 160 to 212° F. to form an aged solids mixture; and separating oil and water from the aged solids mixture to provide a high protein cake comprising at least 35% dry weight crude protein. The high protein cake may optionally be dried, e.g., to a powder. In any embodiments, the high protein cake and compositions thereof includes grain protein, yeast protein, or both grain and yeast protein.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” means up to plus or minus 5% of the particular term. In some embodiments, “about” may mean up to plus or minus 4%, 3%, 2%, or 1% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “barrel screen” refers to a rotating cylinder (barrel) having an inlet and outlet on opposite ends through which mixtures of liquids (e.g., water) and solids may enter and exit the cylinder. The surface of the cylinder includes a wire screen (e.g., woven wire) or porous plate (e.g., perforated plate, or folded, perforated plate) having a mesh size suitable to allow a portion of liquids to flow from the internal space of the cylinder through the screen while retaining at least a portion of the solids within the cylinder. Thus, when the mixture of liquid and solids exit the barrel screen at the outlet, the solids have been much concentrated.

As used herein, “demulsifier” refers to a chemical or composition that acts to reduce or break emulsions, that is separate them into their constituent parts, i.e., oil and water.

As used herein, “air flotation” or DAF refers to a water treatment process whereby a water process stream, e.g., flocculated thin stillage, is exposed to tiny air bubbles that adhere to suspended matter, e.g., flocs including thin stillage solids, and causes the suspended matter to float to the surface of the water where it may be removed, e.g., by skimming. Examples of air flotation processes include but are not limited to dissolved air flotation and induced air flotation.

As used herein, “flocculated thin stillage” refers to thin stillage that includes an effective amount of polymer flocculant such that at least a portion of the thin stillage solids are complexed with the polymer flocculant as flocs.

As used herein, “polysorbates” refer to ethoxylated sorbitans that are esterified with fatty acids (e.g., C10 to C20 fatty acids). Polysorbates may have a range of ethyleneoxy units attached to the sorbitan, e.g., 20-80 or more. Common polysorbates that may be used in the present process as a demulsifier include, without limitation, polysorbate 20, polysorbate 40, polysorbate 60 and polysorbate 80.

As used herein, “stillage solids” refers to both soluble and insoluble solids present in whole stillage or thin stillage, including but not limited to protein, carbohydrates, and other unfermentable gain components that when dried are solid rather than liquid in form. The protein may be grain protein, yeast protein or both grain and yeast protein.

As used herein, “thin stillage” refers to the liquid component separated from whole stillage, typically by centrifugation, as part of an ethanol production process using grain. Thin stillage contains water, oil, and both dissolved and suspended stillage solids, including but not limited to protein and carbohydrates.

As used herein, “polymer flocculant” refers to anionic, cationic and nonionic polymers, including without limitation copolymers, that are capable of flocculating stillage solids from a liquid, e.g., thin stillage.

As used herein, “total suspended solids” refers to the dry weight of undissolved particles in a liquid, e.g., water (where it may be measured according to ASTM SM 2540 D).

The present technology provides processes for efficiently producing a high value, high-protein-content product as part of ethanol production. The process includes the step of subjecting flocculated thin stillage comprising stillage solids and a polymer flocculent to a barrel screen or dissolved air flotation to separate a liquid stream from a solids stream, wherein the solids stream includes flocs of the stillage solids and polymer flocculant. The stillage solids in the flocculated thin stillage may range from 2 wt % to 9 wt %, including 2, 3, 4, 5, 6, 7, 8, and 9 wt % or a range between and including any two of the foregoing values, e.g., 4 wt % to 8 wt %, 2 wt % to 8 wt %, 3 wt % to 8 wt % total solids. Of these solids, a portion may be suspended solids. Thus, the flocculated thin stillage employed may have, e.g., 1, 2, 3, 4, or 5 wt % total suspended solids or a range between and including any two of the foregoing values (e.g., 1-5 wt %, 2-5 wt %, 3-5 wt %, 1-4 wt %, or 1-3 wt % total suspended solids). It will be understood that at least a portion of the suspended solids in the flocculated thin stillage are present as flocs with at least a portion of the polymer flocculant. In any embodiments, the flocculated thin stillage may be from corn.

Any suitable polymer flocculant may be used in any suitable process with the thin stillage to create flocs with the stillage solids. U.S. Pat. Nos. 7,497,955, 7,566,469, and 9,776,105, each of which is incorporated herein by reference in its entirety, describe suitable polymer flocculants and processes in detail. In any embodiments, the polymer flocculant may be an anionic polymer flocculant. Also, in any embodiments, the polymer flocculant may be a polyacrylamide, polyacrylate or copolymer thereof. The copolymers may be random or block copolymers. In any embodiments, the polymer flocculant may be an anionic polymer comprising one or more anionic monomers selected from acrylic acid sodium salt, 2-acrylamido-2-methyl-1-propanesulfonic acid sodium salt and methacrylic acid sodium salt, and optionally one or more acrylamide monomers.

The amount of polymer flocculant present in the thin stillage may range from 20 ppm to 1000 ppm. The amount used will depend on the nature and concentration of solids in the thin stillage as well as the amount of oil and other constituents and the type of polymer flocculant used. Selecting the optimal amount of polymer flocculant for a particular application is within the skill in the art. Examples of suitable concentrations of polymer flocculant include 20, 50, 100, 150, 200, 250, 300, 350, 400, 500, 600 700, 800, 900, and 1000 ppm or a range between and including any two of the foregoing values, e.g., 20 to 350 ppm or 30 to 200 ppm.

Flocculated thin stillage solids have been challenging to remove by traditional settling and filtration methods, limiting their use in protein recovery. They tend to be small (e.g., 5-100 microns in size and “soft.” Settling takes too much liquid volume and too much time, while traditional filtration methods require an impractically large surface area to process the large flow volume of thin stillage and still provide adequate removal of flocculated solids from the stream. One method of separating flocculated solids from thin stillage utilizes dissolved air flotation, induced air flotation or the like. Such flotation methods may be used in the present process. In addition, surprisingly, the present inventors have found that a simple barrel screen effectively separates the flocculated stillage solids without plugging to provide a solids stream with the flocs and a liquid (“clears”) stream. Barrel screens suitable for use in the present process have a screen size suitable to concentrate the flocs without plugging, e.g., 40 mesh, 60 mesh 80 mesh, 100 mesh, and 200 mesh or a range of values between and including any two of the foregoing. In any embodiments, the mesh size ranges from 60 to 100 mesh.

The liquid stream (also referred to herein as “Clears”) exiting the barrel screen (or air flotation process such as DAF) has a significantly reduced amount of total suspended solids, and may have, e.g., 0.1 wt % to 0.9 wt % total suspended solids. In any embodiments, the total suspended solids of the liquid stream may be 0.1, 0.25, 0.5, 0.75 or 0.9 wt % or a range between and including any two of those values, e.g., 0.25 wt % to 0.9 wt % total suspended solids. In contrast, the solids stream exiting the barrel screen or air flotation process may have 2, 3, 4, 5, 6, 7, 8, 9, 10 or more times the concentration of solids contained in the flocculated thin stillage.

As the solids stream from the barrel screen or air flotation (e.g., DAF) may in the form of an emulsion, processes of the present technology may optionally include the step of combining the solids stream with an effective amount of a demulsifier and/or inorganic particles to form a treated solids mixture. In any embodiments, the demulsifier may be selected from the group consisting of polysorbate, ethoxylated alcohols and ethoxylated fatty acids (e.g., C10 to C20 fatty acids) or a mixture of two or more thereof. In any embodiments, the ethoxylation may range from 10 to 100 or more ethoxy units, e.g., 10, 15, 20, 25, 30, 40, 50, 75, 100 or a range between and including any two of the foregoing. In any embodiments, the demulsifier is a polysorbate, for example polysorbate 20, 40, 60 or 80. The effective amount of demulsifier used will vary with the type of emulsion and the nature of the demulsifier. It is within the skill in the art to select an amount that will break the emulsion sufficiently to allow for separation of at least a portion of the constituents. Thus, an effective amount of demulsifier may range from 100 ppm to 1300 ppm. Suitable concentrations may include 100, 200, 300, 400, 500, 750, 1000, or 1300 ppm demulsifier, or a range between and including any two of the foregoing values, e.g., 100 to 1000 ppm or 200 to 1300 ppm. In any embodiments, the demulsifier and/or inorganic particles may be added to the solids stream such as silica particles and/or clay particles to form the treated solids mixture. The inorganic particles may be or include nanoparticles such as silica nanoparticles or clay nanoparticles. Effective concentrations of inorganic particles may range from 100 ppm to 1000 ppm. Examples of effective concentrations include 100, 200, 300, 400, 500, 750, or 1000 ppm inorganic particles, or a range between and including any two of the foregoing values, e.g., 100 to 750 ppm or 200 to 1000 ppm.

In any embodiments, processes of the present technology may include the step of aging the treated solids mixture for a period of time at a temperature of 160 to 212° F. to form an aged solids mixture. The aging step enhances the effects of the demulsifier and/or inorganic particles in breaking any emulsion present in the treated solids mixture. Hence, the period that the treated solids may be aged will vary with the nature of the emulsion. In any embodiments, the treated solids mixture may be aged for 15 minutes to 6 hours. Examples of suitable aging periods include 15 minutes, 30 minutes, 45 minutes, 1 hour, 1.5 hours, 2 hours, 3, hours, 4 hours, 5 hours, or 6 hours or a range between and including any two of the foregoing values, e.g., 15 minutes to 4 hours or 1 hour to 3 hours. The temperature at which the aging takes place should be sufficiently high to assist in the breakage of the emulsion within the time periods given herein, e.g., 160 to 212° F. Optimal temperatures may be selected by those skilled in the art depending on the nature of the emulsion, demulsifier, or inorganic particles being used and may include for example 160, 170, 180, 190, 200, 205, 208, 210, 212° F. or a range between and including any two of the foregoing values, e.g., 160 to 208 or 180 to 210° F. The aging may take place in piping or a tank or other vessel specifically for that purpose, and may be part of a batch process or a continuous process.

The process of the present technology may further include separating oil and water from the aged solids mixture to provide a high protein wet cake (“HPWC”) comprising at least 35% dry weight crude protein and an oil/water mixture (“OWM”). Separation of the OWM from the aged solids mixture to provide the HPWC may be performed by any suitable process such as centrifugation or decanting. For example, the OWM may be separated from the HPWC by subjecting the aged solids mixture to a two-phase vertical or horizontal decanter. Similarly, the present process may further include separating the oil in the OWM from the water (clears) in the OWM using centrifugation, for example a two-phase vertical or horizontal decanter.

The HPWC may be added back to the DDGS in whole or in part or it may be dried to remove residual water and provide a high value protein powder or composition comprising same. This high protein powder (“HPP”) comprising stillage solids includes at least 35 wt % protein. In any embodiments, the amount of protein in the HPSC or HPP may range from 35 wt % to 55 wt % (on a dry weight basis). For example the protein powder comprising stillage solids may have 35, 36, 38, 40, 42, 44, 45, 46, 48, 50, 52, 54, or 55 wt % protein or a range between and including any two of the foregoing values such as 40 wt % to 54 or 55 wt %, or 46 wt % to 54 or 55 wt % protein. The HPWC or HPP may have the same amount or be lower in oil than traditional DDGS, contributing to its higher value as an animal feed. The high protein wet cake or the dry powder may also contain less fiber than traditional DDGS, again contributing to its higher value as an animal feed. For example, the amount of neutral detergent fiber (NDF) in the HPWC or HPP may be less than or equal to 30 wt % or less than or equal to 25 wt %, e.g., about 30, about 27, about 25, about 24, about 22, about 20, about 18, about 16 or about 15 wt % or in a range between and including any two of the foregoing values (e.g., 30-15 wt % or 25-15 wt %). The amount of acid detergent fiber (ADF) may likewise be less in the HPWC or HPP than in traditional DDGS, e.g., less than 10 wt %, or in the range of about 10, about 9, about 8, about 7, about 6, about 5, about 4 wt % or in a range between and including any two of the foregoing values.

The fermentation of grains typically includes the addition of yeast to carry out the process of converting sugars in grain to ethanol. Thus, in any embodiments, the stillage solids may include grain proteins, yeast proteins, or both grain and yeast proteins. In some embodiments with significant amounts of both grain and yeast proteins, processing leading to HPWC may be manipulated to vary the amount of yeast protein in the HPWC. In any embodiments, the amount of yeast proteins may be between about 20 wt % and about 45 wt % of the dry weight of the HPWC, HPP or compositions thereof. For example, in any embodiments, the amount of yeast proteins in the HPWC/HPP/compositions may be about 20, about 25, about 30, about 35, about 40 or about 45 wt % yeast protein or a range between and including any two of the foregoing values. In any embodiments the HPWC/HPP/compositions may include, e.g., 25 to 40 wt % yeast proteins, 30 to 40 wt % or 30 to 45 wt % yeast proteins. The HPWC/HPP/compositions may also include other beneficial yeast components such as amino acids, peptides, carbohydrates, salts, monosodium glutamate, nucleic acids (RNA), enzymes, and cofactors, polysaccharides, e.g., beta-glucans, glycoproteins, mannans, and chitin.

In any embodiment, the process of the present technology may further include separating oil and water from the OWM. The water from this step comprises solids, mostly soluble solids. It may contain, e.g., 1 wt % to 5 wt % solids. This water may be treated as a syrup stream and processed (e.g., evaporated and added to DDGS) according to procedures known in the art. In comparison to traditional methods of treating thin stillage, the present methods use much less energy evaporating water from the thin stillage and provide a new high protein product (in wet cake or powder form) that can be sold at a higher price than traditional DDGS.

The present technology, thus generally described, will be understood more readily by reference to the following examples, which are provided by way of illustration and are not intended to be limiting of the present technology.

### EXAMPLES

### Example 1—Production of a High-Protein Solids Cake from Thin Stillage

The process of the present technology was carried out on thin stillage from a dry grind ethanol process. I.e., the flocculated thin stillage (using GR109 polymer flocculant from Nalco) was processed using the dissolved air flotation method. The resulting solids stream was treated with emulsifier GR812 (Nalco), and aged in a storage tank for 45 minutes. The aged solids mixture was sent to an oil separator, followed by a decanter to separate OWM from HPWC.

### Example 2—Composition of High-Protein Powder Containing Stillage Solids

The HPWC produced in Example 1 was dried and analyzed for the amount of crude protein, NDF, and ADF it contains in accordance with standard methods known in the art. The results are shown in the Table below, compared to the amounts of the same constituents in traditional DDGS, made in the same dry grind ethanol plant. As shown in the table, the HPWC produced has a significantly higher amount of protein and lower amounts of fiber than traditional DDGS.

| | DDGS | HPWC |
|---|---|---|
| Nutritional | (dry weight | (dry weight |
| Constituent | percent) | percent) |
| Crude Protein | 30.6 | 50.0 |
| NDF | 32.7 | 18.1 |
| ADF | 11.3 | 7.0 |

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Further embodiments of processes and compositions are presented in the identified paragraphs below:
A. In any embodiments, the present technology includes a process comprising:
   a. subjecting flocculated thin stillage comprising stillage solids and a polymer flocculent to a barrel screen or dissolved air flotation to separate a liquid stream from a solids stream, wherein the solids stream comprises flocs of the polymer flocculant and the stillage solids;
   b. combining the solids stream with an effective amount of a demulsifier and/or inorganic particles to form a treated solids mixture;
   c. aging the treated solids mixture for a period of time at a temperature of 160 to 212° F. to form an aged solids mixture; and
   d. separating oil and water from the aged solids mixture to provide a high protein wet cake comprising at least 35% dry weight crude protein.
B. The process of paragraph A wherein the thin stillage comprises 4 wt % to 10 wt % total solids and/or 1 wt % to 5 wt % Total Suspended Solids.
C. The process of any one of paragraphs A or B wherein the polymer flocculant is present in the feed stream at concentrations between 20-1000 ppm.
D. The process of paragraph C wherein the polymer flocculant is present in the feed stream at concentrations between 20-350 ppm.
E. The process of any one of paragraphs A-D wherein the flocculated thin stillage comprising stillage solids and a polymer flocculent is subjected to a barrel screen.
F. The process of any one of paragraphs A-E wherein the liquid stream comprises total suspended solids of 0.25 wt % to 0.9 wt %.
G. The process of any one of paragraphs A-F wherein the polymer flocculant is an anionic polymer flocculant.
H. The process of any one of paragraphs 1-G wherein the polymer flocculant is one or more polymers selected from the group consisting of polyacrylamide, polyacrylate, and copolymers thereof.
I. The process of paragraph H wherein the polymer flocculant is an anionic polymer comprising one or more anionic monomers selected from acrylic acid sodium salt, 2-acrylamido-2-methyl-1-propanesulfonic acid sodium salt, methacrylic acid sodium salt, and optionally one or more acrylamide monomers.
J. The process of any one of paragraphs A-I wherein the demulsifer is combined with the solids stream and is selected from the group consisting of polysorbate, ethoxylated alcohols, ethoxylated fatty acids and combinations of two or more thereof.
K. The process of any one of paragraphs A-J wherein the demulsifier is a polysorbate.
L. The process of any one of paragraphs A-K wherein the effective amount of demulsifier is at concentrations between 100-1300 ppm of the solids stream.
M. The process of any one of paragraphs A-L comprising adding an effective amount of inorganic particles to the solids stream.
N. The process of paragraph M wherein the inorganic particles are selected from one or more of silica and clay particles.
O. The process of paragraph M wherein the inorganic particles comprise silica nanoparticles.
P. The process of any one of paragraphs A-O wherein the inorganic particles are present at concentrations between 100-1000 ppm of the treated solids mixture.
Q. The process of any one of paragraphs A-P wherein the treated solids mixture is aged for 15 minutes to 6 hours.
R. The process of any one of paragraphs A-Q wherein the treated solids mixture is aged at a temperature of 180 to 210° F.
S. The process of any one of paragraphs A-R wherein the separation of the oil/water mixture from the protein solids cake comprises centrifugation or decanting.
T. The process of any one of paragraphs A-S wherein the separation of the oil/water mixture from the protein solids cake comprises subjecting the aged solids mixture to a two-phase vertical or horizontal decanter.
U. The process of any one of paragraphs A-T further comprising separating oil and water comprising solids from the oil/water mixture.
V. The process of paragraph U wherein the water comprising solids contains 1 wt % to 5 wt % solids.
W. The process of any one of paragraphs A-V further comprising drying the high protein wet cake to remove residual water and provide a protein powder.
X. The process of any one of paragraphs U-W further comprising separating the oil from the oil/water mixture to provide a water stream (clears).
Y. The high protein wet cake product by the process of any one of paragraphs A-X.
Z A composition comprising stillage solids wherein the composition comprises at least 35 wt % dry weight protein.
AA. The composition of paragraph Z, wherein the composition comprises 35 wt % to 55 wt % dry weight protein.

Other embodiments are set forth in the following claims.

What is claimed is:

1. A process comprising:
a. subjecting flocculated thin stillage comprising stillage solids and a polymer flocculent to a barrel screen or dissolved air flotation to separate a liquid stream from a solids stream, wherein the solids stream comprises flocs of the polymer flocculant and the stillage solids;
b. combining the solids stream with an effective amount of a demulsifier and/or inorganic particles to form a treated solids mixture;
c. aging the treated solids mixture for a period of time at a temperature of 160 to 212° F. to form an aged solids mixture; and
d. separating oil and water from the aged solids mixture to provide a high protein wet cake comprising at least 35% dry weight crude protein.

2. The process of claim 1 wherein the thin stillage comprises 4 wt % to 10 wt % total solids and/or 1 wt % to 5 wt % Total Suspended Solids.

3. The process of claim 1 wherein the polymer flocculant is present in the feed stream at concentrations between 20-1000 ppm.

4. The process of claim 3 wherein the polymer flocculant is present in the feed stream at concentrations between 20-350 ppm.

5. The process of any one of claim 1 wherein the flocculated thin stillage comprising stillage solids and a polymer flocculent is subjected to a barrel screen.

6. The process of any one of claim 1 wherein the liquid stream comprises total suspended solids of 0.25 wt % to 0.9 wt %.

7. The process of any one of claim 1 wherein the polymer flocculant is an anionic polymer flocculant.

8. The process of claim 1 wherein the polymer flocculant is one or more polymers selected from the group consisting of polyacrylamide, polyacrylate, and copolymers thereof.

9. The process of claim 8 wherein the polymer flocculant is an anionic polymer comprising one or more anionic monomers selected from acrylic acid sodium salt, 2-acrylamido-2-methyl-1-propanesulfonic acid sodium salt, methacrylic acid sodium salt, and optionally one or more acrylamide monomers.

10. The process of claim 1 wherein the demulsifer is combined with the solids stream and is selected from the group consisting of polysorbate, ethoxylated alcohols, ethoxylated fatty acids and combinations of two or more thereof.

11. The process of claim 1 wherein the demulsifier is a polysorbate.

12. The process of claim 1 wherein the effective amount of demulsifier is at concentrations between 100-1300 ppm of the solids stream.

13. The process of claim 1 comprising adding an effective amount of inorganic particles to the solids stream.

14. The process of claim 13 wherein the inorganic particles are selected from one or more of silica and clay particles.

15. The process of claim 13 wherein the inorganic particles comprise silica nanoparticles.

16. The process of claim 1 wherein the inorganic particles are present at concentrations between 100-1000 ppm of the treated solids mixture.

17. The process of any claim 1 wherein the treated solids mixture is aged for 15 minutes to 6 hours.

18. The process of claim 1 wherein the treated solids mixture is aged at a temperature of 180 to 210° F.

19. The process of claim 1 wherein the separation of the oil/water mixture from the protein solids cake comprises centrifugation or decanting.

20. The process of claim 1 wherein the separation of the oil/water mixture from the protein solids cake comprises subjecting the aged solids mixture to a two-phase vertical or horizontal decanter.

21. The process of claim 1 further comprising separating oil and water comprising solids from the oil/water mixture.

22. The process of claim 21 wherein the water comprising solids contains 1 wt % to 5 wt % solids.

23. The process of claim 1 further comprising drying the high protein wet cake to remove residual water and provide a protein powder.
